(19) United States
(12) Patent Application Publication
(10) Pub. No.: US 2018/0243340 A1
(43) Pub. Date: Aug. 30, 2018

(54) COMBINATION THERAPY COMBINING CAR + T CELLS WITH APPROPRIATELY TIMED IMMUNODULATORY ANTIBODIES

(71) Applicant: UNIVERSITY OF HOUSTON SYSTEM, Houston, TX (US)

(72) Inventors: Navin Varadarajan, Houston, TX (US); Gabrielle Romain, Houston, TX (US); Laurence Cooper, Houston, TX (US); Harjcct Singh, Houston, TX (US)

(73) Assignee: University of Houston System, Houston, TX (US)

(21) Appl. No.: 15/753,662

(22) PCT Filed: Aug. 23, 2016
(86) PCT No.: PCT/US2016/048135

§ 371 Date: Feb. 20, 2018

Related U.S. Application Data
(60) Provisional application No. 62/209,195, filed on Aug. 24, 2015.

(51) Int. Cl. A61K 35/17; C12N 5/0783; G01N 33/50; C07K 19/00

(52) U.S. Cl. CPC A61K 35/17; C12N 5/0636; A61P 35/00; C07K 19/00; C12N 2501/515; G01N 33/502

(57) ABSTRACT

In some embodiments, the present disclosure pertains to a method of enhancing chimeric antigen receptor expressing T cell function. In some embodiments, the method comprises activating the chimeric antigen receptor expressing T cells. In some embodiments, the method further comprises determining the differential expression of at least one molecule on the chimeric antigen receptor T cells. In some embodiments, the method comprises targeting the at least one molecule. In some embodiments, the present disclosure pertains to a method of treating a tumor in a subject in need thereof. In some embodiments, the method comprises administering to the subject an infusion of chimeric antigen receptor expressing T cells. In some embodiments, the method further comprises determining the differential expression of at least one molecule on the chimeric antigen receptor T cells. In some embodiments, the method comprises targeting the at least one molecule, wherein the molecule is differentially expressed upon activation of the chimeric antigen receptor expressing T cells.

### CROSS REFERENCE TO RELATED APPLICATIONS

This application claims priority to U.S. Provisional Patent Application No. 62/209,195 filed in the United States Patent and Trademark Office on Aug. 24, 2015, the entirety of which is hereby incorporated by reference.

### STATEMENT REGARDING FEDERALLY SPONSORED RESEARCH

This invention was made with government support under the National Institute of Health (NIH) RO1 Grant No. CA174385, and the Cancer Prevention and Research Institute of Texas (CPRIT) Grant No. RP130570. The government has certain rights in the invention.

### FIELD OF INVENTION

This invention pertains to immunotherapeutic methodologies for treating tumors utilizing activated immune cells in combination with either agonist antibodies against immunostimulatory receptors, or antagonist antibodies against immunoinhibitory receptors, present on immune cells to trigger immune cell functions.

### BACKGROUND

It is now generally accepted that immunotherapy has a role in the treatment of cancers, such as but not limited to, advanced melanoma. Research has therefore been focused on the development of immunotherapies, such as gene therapy and immunostimulatory antibodies, that may benefit a larger number of patients. Numerous studies have demonstrated the unprecedented potential of chimeric antigen receptor (CAR)⁺ T cells in the treatment of hematological malignancies. Currently, these strategies only provide responses in some patients. Therefore, there is a need for increasing efficacy of therapies utilizing CAR⁺ T cells that serve to prolong T cell survival as well as induce a strong immune response.

### BRIEF SUMMARY

In some embodiments, the present disclosure pertains to a method of enhancing chimeric antigen receptor expressing T cell function. In some embodiments, the method comprises activating the chimeric antigen receptor expressing T cells. In some embodiments, the method further comprises determining the differential expression of at least one molecule on the chimeric antigen receptor T cells. In some embodiments, the method comprises targeting the at least one molecule. In some embodiments, the molecule is differentially expressed upon activation of the chimeric antigen receptor expressing T cells. In some embodiments, the activation of the chimeric antigen receptor expressing T cells comprises engagement of the chimeric antigen receptor with its corresponding target antigen. In some embodiments, the differentially expressed molecule is identified using RNA-sequencing, microarray analyses, flow cytometry, Nanostring analyses, RNA-FISH, mass cytometry, western blotting, protein staining and microscopy. In some embodiments, the step of targeting the at least one molecule comprises using a protein, antibody, RNA, or a small molecule.

In some embodiments, the present disclosure pertains to a method of selecting chimeric antigen receptor T cells with an enhanced cytotoxic function. In some embodiments, the chimeric antigen receptor T cells are specifically targeted to a cancer cell. In some embodiments, the method comprises obtaining chimeric antigen receptor expressing T cells expressing a chimeric antigen receptor for a target antigen. In some embodiments, the target antigen is expressed on a cancer cell. In some embodiments, the method further comprises activating the chimeric antigen receptor expressing T cells. In some embodiments, the activation of the chimeric antigen receptor expressing T cells is upon engagement of the chimeric antigen receptor with the target antigen. In some embodiments, the method further comprises determining differential expression of at least one molecule expressed on the chimeric antigen receptor expressing T cells following activation. In some embodiments, the method comprises selecting the chimeric antigen receptor expressing T cell population displaying differential expression of the at least one molecule. In some embodiments, the method furger comprises targeting the at least one differentially expressed molecule. In some embodiments, the chimeric antigen receptor expressed is CD19 receptor. In some embodiments the cancer cell is selected from a group consisting of CLL, B-ALL, Leukemia, or Lymphoma.

In some embodiments, the at least one molecule differentially expressed is a co-stimulatory receptor molecule. In some embodiments, the co-stimulatory molecule is selected from a group consisting of OX40, CD137, CD27, CD28, GITR, CD40 and CD30.

In some embodiments, the targeting comprises contacting an agonist of the co-stimulatory molecule with the chimeric antigen receptor expressing T cells. In some embodiments, the step of contacting comprises sequential contacting of the agonist with the chimeric antigen receptor expressing T cells at timed intervals. In some embodiments, the timed intervals are selected from 12 h, 24 h, 48 h, 72 h, 96 h, 1 week, 2 weeks, 3 weeks, 1 month or 2 months.

In some embodiments, the at least one molecule is an immunoinhibitory receptor molecule differentially expressed on the chimeric antigen receptor expressing T cells. In some embodiments, the immunoinhibitory receptor molecule is selected from a group consisting of CTLA4, PD1, LAG3, TIM3, BTLA or CD244, LIGHT.

In some embodiments, the step of targeting comprises contacting an antagonist of the inducible co-stimulatory molecule with the chimeric antigen receptor expressing T cells. In some embodiments, the step of contacting comprises sequential contacting of the antagonist with the chimeric antigen receptor expressing T cells at timed intervals. In some embodiments, the timed intervals are selected from 12 h, 24 h, 48 h, 72 h, 96 h, 1 week, 2 weeks, 3 weeks, 1 month or 2 months.

In some embodiments, the present disclosure pertains to a method of treating a tumor in a subject in need thereof. In some embodiments, the method comprises administering to the subject an infusion of chimeric antigen receptor expressing T cells. In some embodiments, the method further comprises determining the differential expression of at least one molecule on the chimeric antigen receptor T cells. In some embodiments, the method comprises targeting the at least one molecule, wherein the molecule is differentially expressed upon activation of the chimeric antigen receptor expressing T cells.

### BRIEF DESCRIPTION OF THE FIGURES

FIG. 1 shows a summary of transcriptome profiling of killer CAR⁺ T cells at the single cell level showing differential expression of several genes including GZMB, CD137, and TIM3. TIMING assay was run and videos were visually inspected to select group of killer cells and a group of non-killer cells. Then single cell RT-qPCR was performed using the Biomark Fluidigm protocol. Differentially expressed genes are displayed on the Venn diagram for the 3 healthy donor samples, either bearing CAR⁺ with a CD8a hinge domain or an IgG4.

FIG. 2 shows a summary of transcriptome profiling of killer CAR⁺ T cells at the single cell level. Data were normalized between 3 CAR⁺ T cell samples (2 donors, and 2 different hinges), and represented on a volcano plot, with Log 2 of fold changes between mean of all killer cells (aggregated on 3 samples run in 3 independent experiments) and mean of all non-killer cells on x-axis, and p-value after a t-test on the y-axis.

FIG. 3 shows apparent homogeneous expression of many proteins in non-activated CAR⁺ T cells. CAR⁺ T cells were stained with surface antibodies against different receptors found in the gene expression profiling of killer versus non killer cells. Three healthy donor CAR+ T cells were studied, first and second line, are mostly CD4+ CAR⁺ T cells, whereas the third line batch has majority of CD8+ T cells. Most of the studied receptors showed overall homogeneous expression levels, except for CD2, CD69, CD86, CD137, which display some level of heterogeneity.

FIG. 4 shows upregulation of CD137 upon CAR⁺ T cells stimulation. About 100,000 CAR⁺ T cells were co-cultured with 100,000 CD19-expressing EL4 cells for 4 h and immunostained for FACS analysis to observe the change of the molecule presentation at the membrane surface, frequency and mean fluorescence intensity-wise. CAR expression was unchanged, while CD244, CD45RA, TIM3 and PD1 were downregulated upon stimulation. In contrast, CD137 was the only receptor to be upregulated on antigen-activated CAR⁺ T cells.

FIG. 5 illustrates the change at the protein level of several proteins expressed by CAR⁺ T cells upon antigen specific activation with NALM6 target cells, notably the upregulation of CD137, along with CD69 and CD107. T cells were pre-stimulated 6 h with different receptor antibodies/ligands (legend) and then targets were added for the following 6 h, after which cells were stained and acquired by immuno-flow cytometry. Results are expressed as percentages (y-axis) of live CD8+ cells expressing the different markers (x-axis). The upper graph shows results of CAR⁺ T cells non-stimulated and the lower graph displays results upon 6 h target stimulation. Each data point is the average of duplicate wells for 3 donors.

FIG. 6 illustrates the influence of triggering different CAR⁺ T cell receptors (x-axis) upon the changes in expression level of several proteins (right side legend) expressed by CAR⁺ T cells upon antigen specific stimulation with NALM6 target cells. The percentage of dead target cells found in each condition is also depicted (black full circle). T cells were pre-stimulated 6 h with different receptor antibodies/ligands (x-axis) or nothing as control (no Ab) and then targets were added for the following 6 h, after which cells were stained and acquired by immuno-flow cytometry. Results are expressed as percentages (y-axis) of live CD8+ cells expressing the different markers (right side legend). The upper graph shows CAR⁺ T cells non-stimulated and the lower graph displays results upon 6 h target stimulation. Each data point is the average of duplicate wells for 3 donors. The results suggest that CD137 targeting keeps TIM3 and CTLA4 low as compared to no antibody control and all other receptor triggering treatments. In contrast, TIM3 targeting decreases CD69 expression and increases CD137 expression. As regards to adhesion molecules, CD2 and CD58 targeting decrease CD69 expression and CD107 degranulation as well as frequency of dead targets, and this is consistent with higher cytotoxicity.

FIG. 7 illustrates the influence of targeting different CAR⁺ T cell receptors upon CAR⁺ T cells cytotoxicity against NALM6 target cells, as represented by frequencies of dead targets cells (DeadT) amongst all target cells, and by frequencies of degranulating (CD107+) cells amongst CD4+ and CD8+ CAR+ T cells. CAR⁺ T cells were pre-stimulated 6 h with different receptor antibodies/ligands (column) and then target cells were added for the following 6 h, after which cells were stained and acquired by immuno-flow cytometry. Each data point is the average of duplicate wells for 3 donors. The results suggest that CD137, TIM3, and CD244 targeting increase percentages of dead targets and at the same time, increase percentages of degranulating CAR⁺ T cells, which is consistent with higher cytotoxicity, as compared to no Ab control and all other receptor triggering treatments. In contrast, CD2 and CD58 targeting decrease frequencies of dead target cells and frequencies of degranulating (CD107+) CAR⁺ T cells, which is consistent with decreased cytotoxicity.

FIG. 8 illustrates the change at the protein level of several proteins expressed by CAR⁺ T cells upon cytotoxicity (surrogate marker CD107a) induced in presence of NALM6 target cells, notably the upregulation of CD137, CD244 and CTLA4, along with the activation marker CD69. T cells were pre-stimulated 6 h with different receptor antibodies/ligands (right side legend) or nothing as a control (no antibody) and then targets were added for the following 6 h, after which cells were stained and acquired by immuno-flow cytometry. Results are expressed as percentages (y-axis) of cells expressing the different markers (x-axis) among live CD8+ CD107+/− cells (x-axis). Each data point is the average of duplicate wells for 3 donors. The results indicate that upon degranulation, CAR⁺ T cells have increase expression of CD137, CD244 and CTLA4, along with the activation marker CD69, Moreover, CTLA4 expression is increased upon triggering of CD2, TIM3, CD244 and CD58 in comparison to the control (no triggering), but is unchanged, if not decreased, upon CD137 triggering. Stars represent p-values obtained after paired t-test, following the usual convention.

FIG. 9 shows NOD.Cg-Prkdc^{scid}Il2rg^{tm1/wjl}/SzJ (NSG) mice injected intravenously (i.v.) on day 0 with 2×10⁵ NALM-6 tumor cells expressing the firefly luciferase. Groups of mice (5 each) were randomized after tumor engraftment (day 6) and were injected with 10⁷ CAR⁺ T cells on day 6. A control group of mice was left untreated. Anesthetized mice underwent bioluminescent imaging (BLI) in an anterior-posterior position using a Xenogen IVIS 100 series system. The total photon count from NALM-6 xenografts was serially measured using the Living Image program. Mice treated with CAR⁺ T cells expressing CD137L demonstrate better control of tumors. Statistical analysis of photon flux and tumor burden was accomplished using Student's t test.

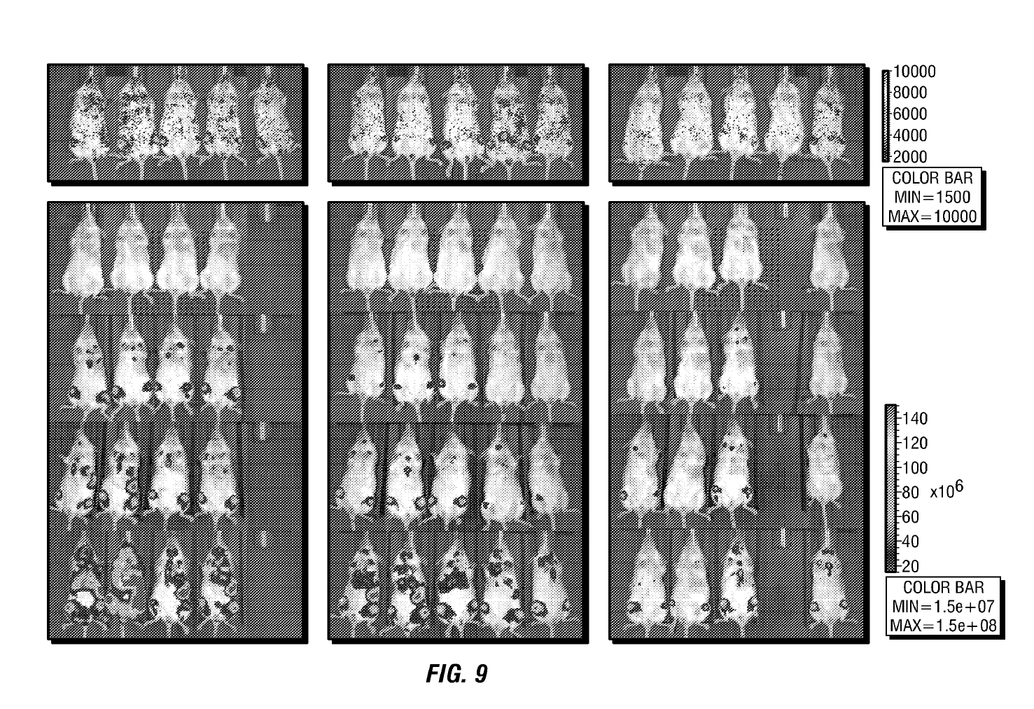

### DETAILED DESCRIPTION

It is to be understood that both the foregoing general description and the following detailed description are exemplary and explanatory only, and are not restrictive of the invention, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that comprise more than one unit unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Unless defined otherwise, all technical and scientific terms used herein have the meaning commonly understood by a person skilled in the art to which the invention belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th ed., R. Reigers et al. (eds.), Springer Verlag (1991); and Hale & Marham, The Harper Collins Dictionary of Biology (1991).

Chimeric antigen receptors (CAR) are hybrid molecules that typically combine the specificity and affinity of single-chain antibodies with selected intracellular signaling domains of the T-cell receptor complex. In some embodiments, the CAR comprises an antigen binding domain, a hinge domain, a transmembrane domain, a co-stimulatory signaling region, and a CD3 zeta signaling domain. In some embodiments, when transduced in T cells, CARs redirect specificity independently of human leukocyte antigen to recognize tumor-associated antigens (TAA). Second- and third-generation CARs include the endodomains for co-stimulatory molecules, and can thus directly endow the different signals needed for T-cell activation upon binding TAA.

Initial data from clinical trials at multiple research centers reporting the adoptive transfer of T cells genetically modified to express a CD19-specific CAR⁺ T cells for the treatment of B-cell malignancies are encouraging, with patients benefiting from complete remissions. T cells genetically modified to express a CD19-specific chimeric antigen receptor (CAR) comprise a heterogeneous population, and their ability to persist and participate in serial killing of tumor cells is a predictor of therapeutic success. These results have accelerated the clinical translation of T cells bearing CARs targeting TAAs other than CD19 for the treatment of hematologic malignancies as well as solid tumors. As a group, these clinical trials differ in the design and specificity of the CARs in the in vitro approach used to manufacture the T cells, the in vivo regimen used to pretreat the recipient, the tumor burden, tumor type, and the T-cell dosing scheme. Thus, drawing conclusions regarding the relative antitumor effects between the populations of bioengineered CAR⁺ T cells is not readily feasible.

One of the hallmarks of a therapeutically successful infusion is the presence of CAR⁺ T cells that can persist to execute multiple tumor cells within the tumor microenvironment. Hence, robust in vivo proliferation of the infused T cells is a key requirement for immunoablation of tumors. Therefore, there exists a need to develop therapeutic approaches that will prolong CAR+ T cell survival and/or proliferation, as well as have the ability to mount a strong immune response.

“Activation,” as used herein, refers to the state of a T cell that has been sufficiently stimulated to induce detectable cellular proliferation. Activation also refers to engagement of the chimeric antigen receptor with its corresponding target antigen. Activation can also be associated with induced cytokine production, and detectable effector functions.

As used herein, an “immunomodulatory molecule or immunomodulatory receptor molecule” or “immunoregulatory molecule” or “immunoregulatory receptor molecule”, or a molecule differentially expressed on chimeric antigen receptor expressing T cells may be an inducible “co-stimulatory molecule” or an immunoinhibitory molecule”. As used herein, an “inducible co-stimulatory molecule” or “immunostimulatory receptor molecule” is a polypeptide expressed on immune cells, including without limitation CAR⁺ T cells, which expression is induced or significantly upregulated during activation of these cells. Co-stimulatory molecule refers to the cognate binding partner on a T cell that specifically binds with a co-stimulatory ligand, thereby mediating a co-stimulatory response by the T cell, such as, but not limited to, proliferation.

Activation of the co-stimulatory molecule enhanced the effector cell function, for example enhancing T cell proliferation, survival, and cytolytic activity, as well as increased IL-2 secretion. Such inducible co-stimulatory molecules are known to those of skill in the art, and include, without limitation, CD137, OX40, GITR, CD30, ICOS, etc.

Agonists of such co-stimulatory molecules, including antibodies that bind to and activate the co-stimulatory molecule, are of interest for the methods of the invention. Many such co-stimulatory molecules are members of the tumor necrosis factor receptor family (TNFR). TNFR-related molecules do not have any known enzymatic activity and depend on the recruitment of cytoplasmic proteins for the activation of downstream signaling pathways.

CD137 is a member of the tumor necrosis factor (TNF) receptor family. Its alternative names are tumor necrosis factor receptor superfamily member 9 (TNFRSF9), 4-1BB, and induced by lymphocyte activation (ILA). Members of this receptor family and their structurally related ligands are important regulators of a wide variety of physiologic processes and play an important role in the regulation of immune responses.

CD137 is expressed by activated NK cells, T and B lymphocytes and monocytes/macrophages. The CD137 gene encodes a 255-amino acid protein with 3 cysteine-rich motifs in the extracellular domain (characteristic of this receptor family), a transmembrane region, and a short N-terminal cytoplasmic portion containing potential phosphorylation sites. Expression in primary cells is strictly activation dependent. The ligand for the receptor is TNFSF9. Human CD137 is reported to bind only to its ligand. Agonists include the native ligand (TNFSF9), aptamers (see McNamara et al. (2008) J. Clin. Invest. 118: 376-386), and antibodies.

The best characterized activity of CD137 is its co-stimulatory activity for activated T cells. Crosslinking of CD137 enhances T cell proliferation, IL-2 secretion, T cell survival, and cytolytic activity. Further, it has been shown to enhance immune activity to eliminate tumors in mice. CD137 has been targeted in many instances to promote immunity against tumors and has shown great promises even in non or poorly immunogenic tumor models in animals and in several tumor settings in humans.

A wide amount of literature demonstrates the benefits of anti-CD137 antibodies when co-administered with various anti-cancer agents such as, IL-2, anti-CTLA4 antibodies, Trp2 Peptides and adjuvant TLR9, and even chemotherapeutic agent 5-flurouracil and radiation therapy. For example, co-administration of anti-CD137 agonist antibodies with NK cells and cytokine induced killer cells (CIK), and dendritic cells. Moreover, inclusion of CD137 in the intracellular domain of, CAR+ T cells or TCR engineered T cells, has also been reported.

As used herein the term “immunoinhibitory molecule” or “immunoinhibitory receptor molecule” is a polypeptide expressed on immune cells, including without limitation CAR+ T cells, which expression is induced or significantly upregulated during activation of these cells. Immunoinhibitory molecules serve as an immune checkpoint, playing an important role in down regulating the immune system by preventing the activation of immune cells or stopping an ongoing immune response. Antagonists of such molecules, including antibodies that bind to and inhibit the function of these molecules, are contemplated for the methods of the invention. Such inducible immunoinhibitory molecules are known to those of skill in the art, and include, without limitation, CTLA4, PD1, LAG3, TIM3, BTLA or CD244, LIGHT.

Agonists of the inducible co-stimulatory molecules include the native ligands, aptamers, antibodies specific for an inducible co-stimulatory molecule that activate the receptor, and derivatives, variants, and biologically active fragments of antibodies that selectively bind to an inducible co-stimulatory molecule. A “variant” polypeptide means a biologically active polypeptide as defined below having less than 100% sequence identity with a native sequence polypeptide. Such variants include polypeptides wherein one or more amino acid residues are added at the N- or C-terminus of, or within, the native sequence; from about one to forty amino acid residues are deleted, and optionally substituted by one or more amino acid residues; and derivatives of the above polypeptides, wherein an amino acid residue has been covalently modified so that the resulting product has a non-naturally occurring amino acid. Ordinarily, a biologically active variant will have an amino acid sequence having at least about 90% amino acid sequence identity with a native sequence polypeptide, preferably at least about 95%, more preferably at least about 99%. The variant polypeptides can be naturally or non-naturally glycosylated, i.e., the polypeptide has a glycosylation pattern that differs from the glycosylation pattern found in the corresponding naturally occurring protein.

Antagonists of the immunoinhibitory molecules include aptamers, antibodies specific for the immunoinhibitory molecule that inhibit the receptor, and derivatives, variants, and biologically active fragments of antibodies that selectively bind to an the immunoinhibitory molecule to suppress its activity. A “variant” polypeptide means a biologically active polypeptide as defined below having less than 100% sequence identity with a native sequence polypeptide. Such variants include polypeptides wherein one or more amino acid residues are added at the N- or C-terminus of, or within, the native sequence; from about one to forty amino acid residues are deleted, and optionally substituted by one or more amino acid residues; and derivatives of the above polypeptides, wherein an amino acid residue has been covalently modified so that the resulting product has a non-naturally occurring amino acid. Ordinarily, a biologically active variant will have an amino acid sequence having at least about 90% amino acid sequence identity with a native sequence polypeptide, preferably at least about 95%, more preferably at least about 99%. The variant polypeptides can be naturally or non-naturally glycosylated, i.e., the polypeptide has a glycosylation pattern that differs from the glycosylation pattern found in the corresponding naturally occurring protein.

Fragments of the ligand or antibodies specific for an inducible co-stimulatory molecule, particularly biologically active fragments and/or fragments corresponding to functional domains are of interest. Fragments of interest will typically be at least about 10 aa to at least about 15 aa in length, usually at least about 50 aa in length, but will usually not exceed about 200 aa in length, where the fragment will have a contiguous stretch of amino acids that is identical to the polypeptide from which it is derived. A fragment “at least 20 aa in length,” for example, is intended to include 20 or more contiguous amino acids from, for example, an antibody specific for CD137, or from TNFSF9. In this context “about” includes the particularly recited value or a value larger or smaller by several (5, 4, 3, 2, or 1) amino acids. The protein variants described herein are encoded by polynucleotides that are within the scope of the invention. The genetic code can be used to select the appropriate codons to construct the corresponding variants. The polynucleotides may be used to produce polypeptides, and these polypeptides may be used to produce antibodies by known methods. A “fusion” polypeptide is a polypeptide comprising a polypeptide or portion (e.g., one or more domains) thereof fused or bonded to heterologous polypeptide.

In preferred embodiments, the inducible co-stimulatory molecule agonist is an antibody. In preferred embodiments, the immunoinhibitory molecule antagonist is an antibody. The term “antibody” or “antibody moiety” is intended to include any polypeptide chain-containing molecular structure with a specific shape that fits to and recognizes an epitope, where one or more non-covalent binding interactions stabilize the complex between the molecular structure and the epitope. Antibodies utilized in the present invention may be polyclonal antibodies, although monoclonal antibodies are preferred because they may be reproduced by cell culture or recombinantly, and can be modified to reduce their antigenicity.

In some embodiments, the present disclosure relates to the discovery that modulation of at least one molecule differentially expressed on the surface of activated CAR⁺ T cells contributes to at least an enhanced antigen-independent activation of the transduced T cells, increased cytotoxicity, increased cytokine secretion, increased cell population expansion of the transduced T cells, increased numbers of progeny of the transduced T cells, and increased persistence of the transduced T cell population both in vitro and in vivo. In some embodiments, the at least one molecule is a co-stimulatory receptor molecule. In some embodiments, the co-stimulatory molecule is induced upon activation of the CAR⁺ T cells. In some embodiments, the at least one molecule is an immunoinhibitory molecule. In some embodiments, the immunoinhibitory molecule is induced upon activation of the CAR⁺ cells.

Thus, in some embodiments, the present disclosure pertains to compositions and methods for treating cancer, including, but not limited, to hematologic malignancies and solid tumors, by the administration of T cells transduced with CARs, specifically targeted to an antigen or a marker expressed by the tumor, i.e., a tumor associated antigen (TAA); and either an agonist-mediated activation of an immunostimulatory receptor molecule or an antagonist-mediated inhibition of an immunoinhibitory molecule, or both, that contributes to increased activation, cytotoxicity, proliferation, and persistence of the transduced T cell population.

In some embodiments, the present disclosure pertains to a method of enhancing CAR⁺ T cell function by appropriately timed stimulation of an inducible co-stimulatory receptor molecule expressed on activated CAR+ T cell. In some embodiments, the stimulation is mediated by an antibody specific for the co-stimulatory receptor molecule. In some embodiments, the at least one inducible co-stimulatory molecule is selected from the group consisting of C D2, OX40, CD137, CD27, CD28, GITR, CD40 and CD30.

In some embodiments, the present disclosure pertains to a method of enhancing CAR⁺ T cell function by appropriately timed inhibition of an immunoinhibitory receptor molecule differentially expressed on activated CAR+ T cell. In some embodiments, the inhibition is mediated by an antibody specific for the immunoinhibitory molecule. In some embodiments, the at least one immunoinhibitory molecule is selected from the group consisting of CTLA4, PD1, LAG3, TIM3, BTLA or CD244, LIGHT.

In some embodiments, the method comprises stimulation of at least one co-stimulatory molecule; and inhibition of at least one immunoinhibitory molecule, differentially expressed on the CAR⁺ T cells. In some embodiments, the CAR+ T cell functions enhanced include cytotoxicity, cytokine secretion, cell survival and cell proliferation.

In some embodiments, the present disclosure relates to a method of enhancing chimeric antigen receptor expressing T cell function. In some embodiments, the method comprises, activating the chimeric antigen receptor expressing T cells. In some embodiments, the method comprises, determining the differential expression of at least one molecule on the chimeric antigen receptor T cells. In some embodiments, the method comprises, targeting the at least one molecule, wherein the at least one molecule is differentially expressed upon activation of the chimeric antigen receptor expressing T cells.

In some embodiments, targeting the at least one differentially expressed molecule comprises targeting at least one secondary molecule. In some embodiments, the at least one secondary molecule is in a regulatory pathway upstream of the at least one differentially expressed molecule. In some embodiments, the at least one secondary molecule is in a regulatory pathway downstream of the at least one differentially expressed molecule. In some embodiments, the at least one secondary molecule is a closely related functional homologue of the at least one differentially expressed molecule. In some embodiments, at least one secondary molecule functions within the same regulatory pathway as the at least one differentially expressed molecule. In some embodiments, the at least one secondary molecule is a known target for an FDA approved drug.

In some embodiments, the activation of the chimeric antigen receptor expressing T cells comprises engagement of the chimeric antigen receptor with its corresponding target antigen.

In some embodiments, the step of determining the differential expression of the at least one molecule comprises using RNA-sequencing, microarray analysis, Nanostring analyses, RNA-FISH, flow cytometry, mass cytometry, western blotting, protein staining, and microscopy.

In some embodiments, the step of targeting the at least one molecule comprises using a protein, antibody, RNA, or a small molecule. In some embodiments, the small molecule is a FDA approved drug.

In some embodiments, the at least one molecule is an inducible co-stimulatory receptor molecule expressed on the chimeric antigen receptor expressing T cells. In some embodiments, the inducible co-stimulatory molecule is selected from a group consisting of CD2, OX40, CD137, CD27, CD28, GITR, CD40 and CD30. In some embodiments, the inducible co-stimulatory receptor molecule is CD137. In some embodiments, the step of targeting comprises contacting an agonist of the inducible co-stimulatory receptor molecule with the chimeric antigen receptor expressing T cells.

In some embodiments of the present disclosure, the chimeric antigen receptor (CAR) encodes for a nucleic acid sequence comprising an antigen binding domain. In some embodiments, the chimeric antigen further receptor encodes for a nucleic acid sequence comprising a transmembrane domain. In some embodiments, the chimeric antigen receptor encodes for a nucleic acid sequence comprising a co-stimulatory domain. In some embodiments, the chimeric antigen further receptor encodes for a nucleic acid sequence a CD3 zeta signaling domain.

In some embodiments, the antigen recognized by the antigen binding domain of the CAR comprises a tumor associated antigen (TAA). In some embodiments, the antigen recognized by the CAR comprises CD19, CD20, CD22, ROR1, human endogenous retroviruses, human immunodeficiency viruses, mesothelin, cancer-associated Tn glycoform of MUC1, EGFRvIII, GD-2, CD33/IL3Ra, PSMA, c-Met, and Glycolipid F77, and any combination thereof.

In some embodiments, the chimeric antigen receptor expressing T cells are effective for treating B cell malignancy, CLL, B-ALL, Leukemia, Lymphoma or solid tumors. In some embodiments, the solid tumors are selected from a group consisting of breast cancer, prostate cancer, bladder cancer, soft tissue sarcoma, lymphomas, esophageal cancer, uterine cancer, bone cancer, adrenal gland cancer, lung cancer, thyroid cancer, colon cancer, glioma; liver cancer, pancreatic cancer, renal cancer, cervical cancer, testicular cancer, head and neck cancer, ovarian cancer, neuroblastoma, and melanoma

In some embodiments of the present disclosure, the at least one molecule is an immunoinhibitory receptor molecule expressed on the chimeric antigen receptor expressing T cells. In some embodiments, the immunoinhibitory receptor molecule is selected from a group consisting of CTLA4, PD1, LAG3, TIM3, BTLA or CD244, LIGHT. In some embodiments, the step of targeting comprises contacting an antagonist of the immunoinhibitory receptor molecule with the chimeric antigen receptor expressing T cells.

In some embodiments of the present disclosure, the activation of the chimeric antigen receptor expressing T cells is measured by assessing chimeric antigen receptor T cell-mediated cytotoxicity, cytokine secretion, cell survival, phenotypic markers, calcium signaling, glycolytic activation, and cell proliferation. In some embodiments, the cytotoxicity is assessed by staining for degranulation marker, granzyme B expression, perforin expression, or microscopy. In some embodiments, the cytokine secretion is assessed by ELISpot, intracellular staining, cytokine catching assay, or single-cell cytokine assays. In some embodiments, the phenotypic markers comprise CD25, CD69, CD137, or CD154. In some embodiments, the cell survival and proliferation is determined using CFSE dilution or Annexin V staining assays. In some embodiments, the calcium signaling is measured using microscopy or flow cytometry using appropriate dyes. In some embodiments, the glycolytic activation is measured using Seahorse assay.

In some embodiments of the present disclosure, the activation is mediated by coculture with autologous tumor cells, cell lines, plate bound antibody against CD3, beads coated with antibody against CD3/CD28. In some embodiments, the activation is measured in the presence of immunosuppressive molecules including TGFβ, IL-10, adenosine, kynurenine, or lactate. In some embodiments, the chimeric antigen further receptor encodes for a nucleic acid sequence the activation is measured under nutrient starvation including glucose limitation, addition of oncometabolites, and amino acid limitation.

In some embodiments, the step of targeting the at least one molecule differentially expressed on the chimeric antigen receptor expressing T cells comprises contacting an antagonist of the inducible immunoinhibitory receptor molecule with the chimeric antigen receptor expressing T cells. In some embodiments, the step of contacting comprises sequential contacting of the antagonist with the chimeric antigen receptor expressing T cells at timed intervals. In some embodiments, the timed intervals are selected from 12 h, 24 h, 48 h, 72 h, 96 h, 1 week, 2 weeks, 3 weeks, 1 month or 2 months.

In some embodiments, the present disclosure pertains to a method of selecting a chimeric antigen receptor T cell population with an enhanced cytotoxic function. In some embodiments, the method comprises obtaining chimeric antigen receptor expressing T cells expressing a chimeric antigen receptor for a target antigen. In some embodiments, the target antigen is expressed on a cancer cell. In some embodiments, the step of obtaining comprises harvesting T cells from the subject. In some embodiments, the method further comprises engineering the T cells to express at least one chimeric antigen receptor. In some embodiments, the at least one chimeric antigen receptor expressed binds to at least one target antigen. In some embodiments, the target antigen is expressed on a cancer cell. In some embodiments, the method further comprises activating the chimeric antigen receptor expressing T cells. In some embodiments, the activation of the chimeric antigen receptor expressing T cells comprises contacting the chimeric antigen receptor expressing T cells with the target antigen. In some embodiments, the activation of the chimeric antigen receptor expressing T cells comprises engagement of the antigen binding domain of the chimeric antigen receptor with the target antigen.

In some embodiments, the method further comprises determining expression of at least one molecule differentially expressed on the chimeric antigen receptor expressing T cells following activation. In some embodiments, the method comprises selecting the chimeric antigen receptor expressing T cell population displaying differential expression of the at least one molecule.

In some embodiments, the method comprises targeting the at least one molecule. In some embodiments, targeting the at least one differentially expressed molecule comprises targeting at least one secondary molecule. In some embodiments, the at least one secondary molecule is in a regulatory pathway upstream of the at least one differentially expressed molecule. In some embodiments, the at least one secondary molecule is in a regulatory pathway downstream of the at least one differentially expressed molecule. In some embodiments, the at least one secondary molecule is a closely related functional homologue of the at least one differentially expressed molecule. In some embodiments, the at least one secondary molecule functions within the same regulatory pathway as the at least one differentially expressed molecule. In some embodiments, the at least one secondary molecule is a known target for an FDA approved drug.

In some embodiments, the at least one differentially expressed molecule is a co-stimulatory receptor molecule. In some embodiments, the step of targeting comprises contacting the selected chimeric antigen receptor expressing T cells with an agonist of the co-stimulatory molecule. In some embodiments, the at least one differentially molecule is an immunoinhibitory receptor molecule. In some embodiments, the step of targeting comprises contacting the selected chimeric antigen receptor expressing T cells with an antagonist of the immunoinhibitory molecule. In some embodiments, the chimeric antigen receptor expressed is CD19 receptor. In some embodiments the cancer cell is selected from a group consisting of CLL, B-ALL, Leukemia, or Lymphoma.

In some embodiments, the step of targeting the at least one molecule differentially expressed on the chimeric antigen receptor cells enhances the chimeric antigen receptor expressing T cell functions. In some embodiments, the functions enhanced comprise cytotoxicity, cytokine secretion, cell survival and cell proliferation.

In some embodiments, the present disclosure pertains to a method of treating a tumor in a subject in need thereof. In some embodiments, the method comprises, obtaining chimeric antigen receptor expressing T cells expressing a chimeric antigen receptor targeting at least one tumor associated antigen. In some embodiments, the method further comprises administering to the subject an infusion of the chimeric antigen receptor expressing T cells. In some embodiments, the method comprises determining the differential expression of at least one molecule on the chimeric antigen receptor T cells. In some embodiments, the method comprises targeting the at least one differentially expressed molecule, wherein the molecule is differentially expressed upon activation of the chimeric antigen receptor expressing T cells.

In some embodiments, targeting the at least one differentially expressed molecule comprises targeting at least one secondary molecule. In some embodiments, the at least one secondary molecule is in a regulatory pathway upstream of the at least one differentially expressed molecule. In some embodiments, the at least one secondary molecule is in a regulatory pathway downstream of the at least one differentially expressed molecule. In some embodiments, the at least one secondary molecule is a closely related functional homologue of the at least one differentially expressed molecule. In some embodiments, the at least one secondary molecule functions within the same regulatory pathway as the at least one differentially expressed molecule. In some embodiments, the at least one secondary molecule is a known target for an FDA approved drug.

In some embodiments, the activation of the chimeric antigen receptor expressing T cells comprises engagement of the chimeric antigen receptor with its corresponding tumor associated antigen.

In some embodiments, the step of determining the differential expression of the at least one molecule comprises using RNA-sequencing, microarray analysis, flow cytometry, Nanostring analyses, RNA-FISH, mass cytometry, western blotting, protein staining, and microscopy.

In some embodiments, the step of targeting the at least one molecule comprises using a protein, antibody, RNA, or a small molecule. In some embodiments, the small molecule is a FDA approved drug.

In some embodiments, the at least one differentially expressed molecule is a co-stimulatory receptor molecule. In some embodiments, the co-stimulatory molecule is selected from a group consisting of CD2, OX40, CD137, CD27, CD28, GITR, CD40 and CD30. In some embodiments, the co-stimulatory receptor molecule is CD137. In some embodiments, the step of targeting comprises contacting an agonist of the co-stimulatory receptor molecule with the chimeric antigen receptor expressing T cells.

In some embodiments, the step of targeting comprises administering to the subject an agonist of the co-stimulatory molecule. In some embodiments, the agonist is administered at the same time, before, or after the administration of the infusion of CAR+ T cells. In some embodiments, the agonist is administered at timed intervals following the infusion of chimeric antigen receptor expressing T cells. In some embodiments, the timed intervals are selected from 12 h, 24 h, 48 h, 72 h, 96 h, 1 week, 2 weeks, 3 weeks, 1 month or 2 months.

In some embodiments the at least one differentially expressed molecule is an immunoinhibitory receptor molecule. In some embodiments, the immunoinhibitory receptor molecule is selected from a group consisting of CTLA4, PD1, LAGS, TIM3, BTLA or CD244, LIGHT. In some embodiments, the step of targeting comprises contacting an antagonist of the immunoinhibitory receptor molecule with the chimeric antigen receptor expressing T cells.

In some embodiments, the step of targeting comprises administering to the subject an antagonist of the immunoinhibitory receptor molecule. In some embodiments, the antagonist is administered at the same time, before, or after the administration of the infusion of CAR+ T cells. In some embodiments, the antagonist is administered at timed intervals following the infusion of chimeric antigen receptor expressing T cells. In some embodiments, the timed intervals are selected from 12 h, 24 h, 48 h, 72 h, 96 h, 1 week, 2 weeks, 3 weeks, 1 month or 2 months.

In some embodiments, the chimeric antigen receptor encodes for a nucleic acid sequence comprising an antigen binding domain. In some embodiments, the chimeric antigen receptor encodes for a nucleic acid sequence comprising a transmembrane domain. In some embodiments, the chimeric antigen receptor encodes for a nucleic acid sequence comprising a co-stimulatory domain. In some embodiments, the chimeric antigen receptor encodes for a nucleic acid sequence comprising a CD3 zeta signaling domain.

In some embodiments, the antigen binding domain recognizes and binds to a tumor associated antigen expressed on the tumor. In some embodiments, the tumor associated antigen recognized by the antigen binding domain of the CAR comprises CD19, CD20, CD22, ROR1, human endogenous retroviruses, human immunodeficiency viruses, mesothelin, human endogenous retroviruses, human immunodeficiency viruses, cancer-associated Tn glycoform of MUC1, EGFRvIII, GD-2, CD33/IL3Ra, PSMA, c-Met, and Glycolipid F77, and any combination thereof.

In some embodiments, the chimeric antigen receptor expressing T cells are effective for treating B cell malignancy, CLL, B-ALL, Leukemia, Lymphoma or solid tumors. In some embodiments, the solid tumors are selected from a group consisting of breast cancer, prostate cancer, bladder cancer, soft tissue sarcoma, lymphomas, esophageal cancer, uterine cancer, bone cancer, adrenal gland cancer, lung cancer, thyroid cancer, colon cancer, glioma; liver cancer, pancreatic cancer, renal cancer, cervical cancer, testicular cancer, head and neck cancer, ovarian cancer, neuroblastoma, and melanoma.

In some embodiments, the activation of chimeric antigen receptor expressing T cell is measured by cytotoxicity, cytokine secretion, phenotypic markers, cell survival and cell proliferation, calcium signaling or glycolytic activation.

In some embodiments the subject is a mammal. In some embodiments, the subject is a human.

In some embodiments, the method further comprises administration of a chemotherapeutic agent to the subject in need thereof.

Useful chemotherapeutic agents include nitrogen mustards, nitrosorueas, ethyleneimine, alkane sulfonates, tetrazine, platinum compounds, pyrimidine analogs, purine analogs, antimetabolites, folate analogs, anthracyclines, taxanes, vinca alkaloids, topoisomerase inhibitors and hormonal agents. Exemplary chemotherapy drugs are Actinomycin-D, Alkeran, Ara-C, Anastrozole, Asparaginase, BiCNU, Bicalutamide, Bleomycin, Busulfan, Capecitabine, Carboplatin, Carboplatinum, Carmustine, CCNU, Chlorambucil, Cisplatin, Cladribine, CPT-I 1, Cyclophosphamide, Cytarabine, Cytosine arabinoside, Cytoxan, Dacarbazine, Dactinomycin, Daunorubicin, Dexrazoxane, Docetaxel, Doxorubicin, DTIC, Epirubicin, Ethyleneimine, Etoposide, Floxuridine, Fludarabine, Fluorouracil, Flutamide, Fotemustine, Gemcitabine, Herceptin, Hexamethylamine, Hydroxyurea, Idarubicin, Ifosfamide, Irinotecan, Lomustine, Mechlorethamine, Melphalan, Mercaptopurine, Methotrexate, Mitomycin, Mitotane, Mitoxantrone, Oxaliplatin, Paclitaxel, Pamidronate, Pentostatin, Plicamycin, Procarbazine, Rituximab, Steroids, Streptozocin, STI-571, Streptozocin, Tamoxifen, Temozolomide, Teniposide, Tetrazine, Thioguanine, Thiotepa, Tomudex, Topotecan, Treosulphan, Trimetrexate, Vinblastine, Vincristine, Vindesine, Vinorelbine, VP-16, and Xeloda. Useful cancer chemotherapeutic agents also include alkylating agents, such as Thiotepa and cyclosphosphamide; alkyl sulfonates such as Busulfan, Improsulfan and Piposulfan; aziridines such as Benzodopa, Carboquone, Meturedopa, and Uredopa; ethylenimines and methylamelamines including altretamine, triethylenemelamine, trietylenephosphoramide, triethylenethiophosphaoramide and trimethylolomelamine; nitrogen mustards such as Chlorambucil, Chlornaphazine, Cholophosphamide, Estramustine, Ifosfamide, mechlorethamine, mechlorethamine oxide hydrochloride, Melphalan, Novembiehin, Phenesterine, Prednimustine, Trofosfamide, uracil mustard; nitroureas such as Carmustine, Chlorozotocin, Fotemustine, Lomustine, Nimustine, and Ranimustine; antibiotics such as Aclacinomysins, Actinomycin, Authramycin, Azaserine, Bleomycins, Cactinomycin, Calicheamicin, Carabicin, Carminomycin, Carzinophilin, Chromoinycins, Dactinomycin, Daunorubicin, Detorubicin, 6-diazo-5-oxo-L-norleucine, Doxorubicin, Epirubicin, Esorubicin, Idambicin, Marcellomycin, Mitomycins, mycophenolic acid, Nogalamycin, Olivomycins, Peplomycin, Potfiromycin, Puromycin, Quelamycin, Rodorubicin, Streptonigrin, Streptozocin, Tubercidin, Ubenimex, Zinostatin, and Zorubicin; anti-metabolites such as Methotrexate and 5-fluorouracil (5-FU); folic acid analogues such as Denopterin, Methotrexate, Pteropterin, and Trimetrexate; purine analogs such as Fludarabine, 6-mercaptopurine, Thiamiprine, and Thioguanine; pyrimidine analogs such as Ancitabine, Azacitidine, 6-azauridine, Carmofur, Cytarabine, Dideoxyuridine, Doxifluridine, Enocitabine, Floxuridine, and 5-FU; androgens such as Calusterone, Dromostanolone Propionate, Epitiostanol, Rnepitiostane, and Testolactone; anti-adrenals such as aminoglutethimide, Mitotane, and Trilostane; folic acid replenisher such as frolinic acid; aceglatone; aldophosphamide glycoside; aminolevulinic acid; Amsacrine; Bestrabucil; Bisantrene; Edatraxate; Defofamine; Demecolcine; Diaziquone; Elfornithine; elliptinium acetate; Etoglucid; gallium nitrate; hydroxyurea; Lentinan; Lonidamine; Mitoguazone; Mitoxantrone; Mopidamol; Nitracrine; Pentostatin; Phenamet; Pirarubicin; podophyllinic acid; 2-ethylhydrazide; Procarbazine; PSK®; Razoxane; Sizofrran; Spirogermanium; tenuazonic acid; triaziquone; 2, 2′,2″-trichlorotriethylamine; Urethan; Vindesine; Dacarbazine; Mannomustine; Mitobronitol; Mitolactol; Pipobroman; Gacytosine; Arabinoside (“Ara-C”); cyclophosphamide; thiotEPa; taxoids, e.g., Paclitaxel (TAXOL®, Bristol-Myers Squibb Oncology, Princeton, N.J.) and Doxetaxel (TAXOTERE®, Rhone-Poulenc Rorer, Antony, France); Chlorambucil; Gemcitabine; 6-thioguanine; Mercaptopurine; Methotrexate; platinum analogs such as Cisplatin and Carboplatin; Vinblastine; platinum; etoposide (VP-16); Ifosfamide; Mitomycin C; Mitoxantrone; Vincristine; Vinorelbine; Navelbine; Novantrone; Teniposide; Daunomycin; Aminopterin; Xeloda; Ibandronate; CPT-11; topoisomerase inhibitor RFS 2000; difluoromethylornithine (DMFO); retinoic acid; Esperamicins; Capecitabine; and pharmaceutically acceptable salts, acids or derivatives of any of the above. Also included are anti-hormonal agents that act to regulate or inhibit hormone action on tumors such as anti-estrogens including for example Tamoxifen, Raloxifene, aromatase inhibiting 4(5)-imidazoles, 4 Hydroxytamoxifen, Trioxifene, Keoxifene, Onapristone, And Toremifene (Fareston); and anti-androgens such as Flutamide, Nilutamide, Bicalutamide, Leuprolide, and Goserelin; and pharmaceutically acceptable salts, acids or derivatives of any of the above.

In some embodiments the method further comprises administration of at least one cytokine to the subject in need thereof.

Examples of cytokines that may be used in conjunction with the methods of the present disclosure include lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet growth factor; transforming growth factors (TGFs) such as TGF-α and TGF-β; insulin-like growth factor-I and -II; erythropoietin (EPO); osteoinductive factors; interferons such as interferon-α, -β and -γ; colony stimulating factors (CSFs) such as macrophage-CSF (M-CSF); granulocyte-macrophage-CSF (GM-CSF); and granulocyte-CSF (GCSF); interleukins (ILs) such as IL-I, IL-Ia, IL-2, IL-3, IL-4, IL-5, IL-6, IL-7, IL-8, IL-9, IL-I 1, IL-12, IL-15; a tumor necrosis factor such as TNF-α or TNF-β; and other polypeptide factors including LIF and kit ligand (KL). As used herein, the term cytokine includes proteins from natural sources or from recombinant cell culture and biologically active equivalents of the native sequence cytokines.

In some embodiments the method further comprises administration of radiation therapy to the subject in need thereof.

In some embodiments, the method of treating a tumor in a subject in need thereof comprises obtaining chimeric antigen receptor expressing T cells. In some embodiments, the chimeric antigen receptor expressing T cells obtained are engineered to express at least one chimeric antigen receptor specific for at least one tumor associated antigen. In some embodiments, the method further comprises activating the chimeric antigen receptor expressing T cells. In some embodiments, the step of activation comprises contacting the chimeric antigen receptor expressing T cells with the tumor cells. In some embodiments, the step of contacting comprises co-culturing the chimeric antigen receptor expressing T cells with the tumor cells in vitro.

In some embodiments, the method comprises determining the differential expression of at least one molecule on the chimeric antigen receptor T cells. In some embodiments, the method comprises targeting the at least one molecule differentially expressed upon activation of the chimeric antigen receptor expressing T cells.

In some embodiments, targeting the at least one differentially expressed molecule comprises targeting at least one secondary molecule. In some embodiments, the at least one secondary molecule is in a regulatory pathway upstream of the differentially expressed molecule. In some embodiments, the at least one secondary molecule is in a regulatory pathway downstream of the differentially expressed molecule. In some embodiments, the at least one secondary molecule is a closely related functional homologue of the differentially expressed molecule. In some embodiments, the at least one secondary molecule functions within the same regulatory pathway as the differentially expressed molecule. In some embodiments, the at least one secondary molecule is selected based on being a known target for an FDA approved drug.

In some embodiments, the step of activating comprises administering the chimeric antigen receptor expressing T cells to the subject. In some, embodiments, the method further comprises subsequently redrawing blood (or have an apheresis performed) from the subject, and selecting a population of the chimeric antigen receptor T cells. In some embodiments, the step of selecting a population of the chimeric antigen receptor expressing T cells is based on determining expression of at least one molecule differentially expressed on the chimeric antigen receptor expressing T cells. In some embodiments, the at least one molecule is differentially expressed upon activation of the chimeric antigen receptor expressing T cells. In some embodiments, the method comprises, expanding the selected population expressing the at least one differentially expressed molecule.

In some embodiments, the method comprises targeting the at least one differentially expressed molecule on the chimeric antigen receptor expressing T cells.

In some embodiments, targeting the at least one differentially expressed molecule comprises targeting at least one secondary molecule. In some embodiments, the at least one secondary molecule is in a regulatory pathway upstream of the differentially expressed molecule. In some embodiments, the secondary molecule is in a regulatory pathway downstream of the differentially expressed molecule. In some embodiments, the at least one secondary molecule is a closely related protein homologue of the differentially expressed molecule. In some embodiments, the method comprises infusing the subject with the activated and expanded chimeric antigen receptor expressing T cells. In some embodiments, the targeting comprises administering to the subject a modulator of the at least one differentially expressed molecule. In some embodiments, the method comprises administering to the subject a modulator of at least one second molecule.

In some embodiments, a modulator is a protein, an antibody, a RNA, or a small molecule. In some embodiments, a modulator is an agonist of the molecule. In some embodiments, the modulator is an antagonist of the molecule.

In some embodiments, the modulator is administered at the same time, before, or after the administration of the infusion of chimeric antigen receptor expressing T cells. In some embodiments, the modulator is administered at timed intervals following the infusion of chimeric antigen receptor expressing T cells. In some embodiments, the timed intervals are selected from 12 h, 24 h, 48 h, 72 h, 96 h, 1 week, 2 weeks, 3 weeks, 1 month or 2 months.

The compositions disclosed herein may be administered to a subject, such as human, via any suitable administration method in order to treat the tumor. The particular method employed for a specific application is determined by the attending physician. The majority of therapeutic applications may involve some type of parenteral administration, which includes intravenous (i.v.), intramuscular (i.m.) and subcutaneous (s.c.) injection.

Administration of the compositions of the present disclosure may be systemic or local. Local intravascular delivery may be used to bring a therapeutic substance to the vicinity of a known lesion by use of guided catheter system, such as a CAT-scan guided catheter. General injections, such as a bolus i.v. injection or continuous/trickle-feed i.v. infusion are typically systemic.

For intravenous administration, the compositions disclosed herein may be formulated as a suspension in any suitable aqueous carrier vehicle. A suitable pharmaceutical carrier is one that is non-toxic to the recipient at the dosages and concentrations employed and is compatible with other ingredients in the formulation.

### Applications and Advantages

In some embodiments, the present disclosure pertains to promoting overall persistence and efficacy of adoptive cell therapies using CAR+ T cells by stimulating CD137 in parallel with infusion of CAR+ T cells in a subject in need thereof. The methods and compositions contemplated herein are not limited to treating only CD19 expressing B cell malignancies but are also applicable for the treatment of other kinds of cancer, including solid tumors. The methods and compositions disclosed herein also contemplate using antibody agonists of immunostimulatory receptors or antagonists of inhibitory receptors.

The advantage of the methods and compositions disclosed herein lies in the ease and simplicity of delivering or providing both therapeutics, CAR+ T cells and the contemplated antibody, for stimulating or inhibiting specific receptors on the CAR+ T cells, to a subject in need thereof.

Fully humanized agonist antibodies against CD-137 receptor, including BMS-666513 have been tested in phase I and phase II clinical trials in patients suffering from B cell malignancies, and solid tumors like melanoma, renal cell carcinoma, ovarian cancer, and non-small cell lung cancer demonstrating favorable safety profiles and encouraging results.

Applicants have demonstrated CD19-specific CAR+ T cells function with CD19 expressing target cells and shown several characteristics that define the cells capable of cytotoxicity in vitro: this includes characteristics related to motility, time of contact, secretion of IFN-γ, and ability to resist activation induced cell death. Furthermore, with the aid of gene expression profiling, Applicants have been able to identify and sort cytotoxic cells from non-cytotoxic cells in real-time. In some embodiments, cytotoxic CAR+ T cells had an increased basal level expression for CD137. In some embodiments, the CD137 expression was significantly upregulated on the surface of the cytotoxic CAR+ T cells, following antigen-specific stimulation of CD137.

### Additional Embodiments

Reference will now be made to various embodiments of the present disclosure and experimental results that provide support for such embodiments. Applicants note that the disclosure herein is for illustrative purposes only and is not intended to limit the scope of the claimed subject matter in any way.

### Example 1

Human subject statements. All work outlined in this report was performed according to protocols approved by the Institutional Review Boards at the University of Houston and the University of Texas MD Anderson Cancer Center.

### Example 2

Cell Lines

The human pre-B cell line NALM-6 (ATCC), T-cell lymphoma EL-4 (ATCC), and modified CD19⁺EL-4 cells were cultured as recommended by ATCC. The cell lines were routinely tested to ensure that they were free of Mycoplasma contamination and flow cytometry was used to confirm the expression of CD19.

### Example 3

Genetic Modification and Propagation of Cells

Peripheral blood mononuclear cells (PBMC) from healthy volunteers were electroporated using Nucleofector II (Amaxa/Lonza) with DNA plasmids encoding for the Sleeping Beauty (SB) system enforcing the expression of a second generation CD19-specific chimeric antigen receptor (CAR) (designated CD19RCD28) that activates T cells via a chimeric CD3 and CD28 endodomain. The approach to producing the CAR⁺ T cells mirrors our manufacture in compliance with current good manufacturing practice for human application (clinicaltrial.gov NCT00968760 and NCT01497184 under INDs 14193 and 14577, respectively). For single-cell analysis, frozen CAR⁺ T cells were revived and restimulated with irradiated K562 aAPC and cytokines before using them in experiments.

### Example 4

Flow Cytometry

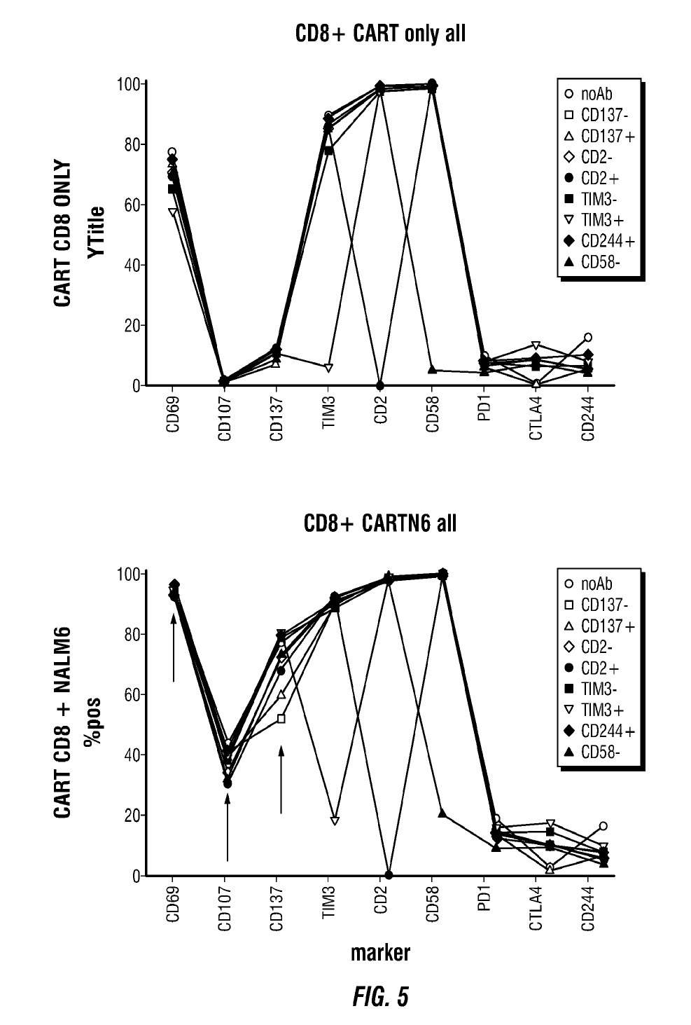

For the experiment described in FIGS. 5-8, the following method was employed. Purified antibodies targeting T cell receptors and CD137L protein (R&D Systems) were coated in 100 μL PBS at 5 μg/mL at 4° C. for 24 h on U-bottom 96-well plates. After emptying these solutions, plates were loaded with 100,000 unstained CAR+ T cells/well, briefly spun down at 100×g for 1 min, and CAR+ T cells were pre-incubated for 6 h in complete culture medium RPMI 10% FBS. Then target cells (EL4CD19+ or NALM6) were stained for 2 min with 1 μM of PKH Green (Sigma-Aldrich) following manufacturer's recommendations, and loaded at 100,000 cells/well onto the CAR+ T cells, mixed thoroughly by pipetting, and settled at the bottom of the plate by brief centrifugation. Each condition was tested using duplicate wells, 3 donors of CAR+ T cells, and controlled by similar wells but without target cells. Co-culture plates were in a cell culture incubator for 6 h at 37° C. After 6 h, cells were pelleted and washed in PBS twice, stained for viability using 0.5 μL/mL of Live/Dead Aqua (LifeTechnologies) for 20 min at 4° C., washed twice in PBS, and stained for cell surface markers using antibodies from Biolegend, BD Biosciences and R&D Systems: CD2-BUV395 (clone RPA-2.10), PD1-BV421 (clone EH12.1), CD58-BV605 (clone 1C3), CD107a-BV786 (cloneH4A3), CD137-PerCP-Cy5 (clone 4B4-1), TIM3-PE (clone FAB2365P), CD69-PE-Dazzle594 (clone FN50), CTLA4-PE-Cy7 (clone L3D10), CD244-APC (clone 2-69) CD4-APC-Cy7 (clone OKT4) CD8-AF700 (301028). After 20 min staining at 20° C., cells were fixed using 1× Nuclear factor fixation buffer (Biolegend) for 20 minutes at 4° C. Cells were subsequently suspended in PBS 2% FBS and acquired on a BD Fortessa X20 or a BD FACSJazz flow cytometer, and analyzed using FCS Express/FlowJo as previously described.

Flow Cytometry

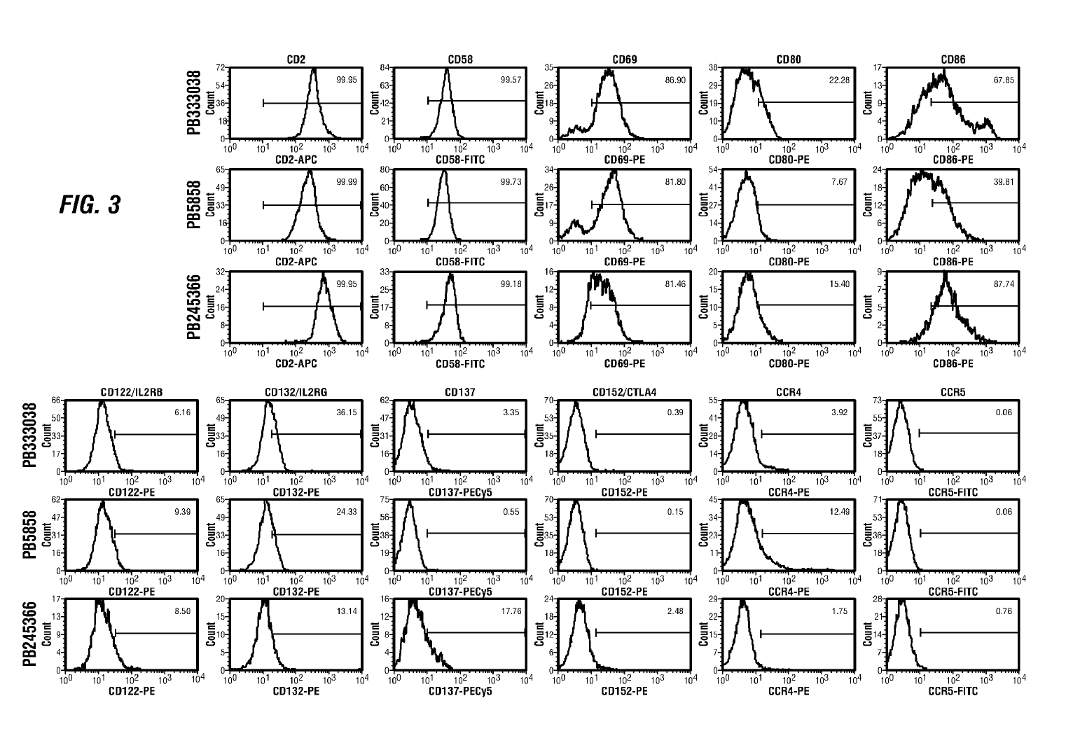

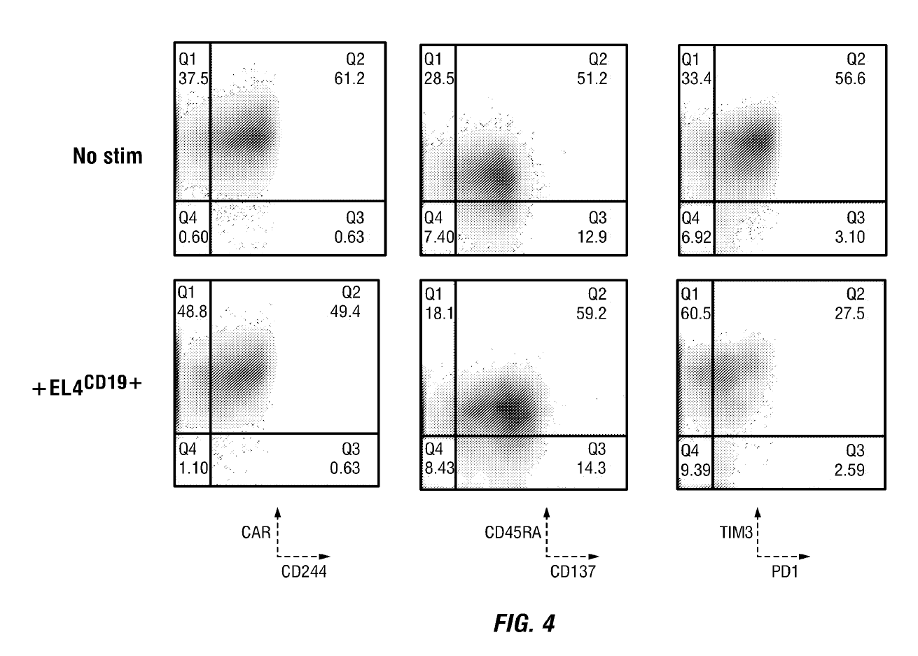

FIGS. 3 and 4 have used slightly modified protocols. In FIG. 3, CAR+ T cells were stained without pre-stimulation with target cells using antibodies conjugated to FITC, PE, APC or PE-Cy5, all purchased from BD Biosciences. In FIG. 4, a similar protocol as described in previous paragraph except that target cells were CD19+EL4 cells.

### Example 5

Single cell cytotoxicity assay (TIMING) Nanowell array fabrication and the corresponding cytotoxicity assay to interrogate effector-target interaction at single-cell level were performed as described previously (Liadi et al, 2012, Journal of Visual Experiments). Nanowell grids were fixed in position on a 50 mm diameter, glass bottom Petri dishes (Ted Pella). CAR⁺ T cells labeled with 1 μmol/L of red fluorescent dye, PKH26 (Sigma) and CD19+ EL4 target cells labeled with 1 μmol/L of green fluorescent dye PKH67 were co-loaded onto nanowell arrays at a concentration of 10⁶ cells/mL. Images were acquired on a Carl Zeiss Axio Observer fitted with a Hamamatsu scientific C-MOS Orca-flash camera using a 20× 0.8 NA objective. Automated image acquisition of the entire chip was performed at regular time interval (5 min) for 3 h and apoptosis was identified by staining with Annexin V conjugated to Alexa-647 (Life Technologies).

### Example 6

Single Cell Gene Expression Profiling Using Multiplexed RT-qPCR

After TIMING run exactly as described above, cells on the nanowell array were carefully washed 3 times with cold PBS (4° C.), and were kept at 4° C. until retrieval. Time-lapse sequences were manually analyzed to identify live killer and non-killer CAR+ T cells in wells containing exactly 1 CAR T cell and 1 to 4 CD19+EL4 target cells. The cells in wells of interest were individually collected using an automated micro-manipulating system (CellCelector, ALS) and deposited in nuclease free microtubes containing 5 μL of 2× CellsDirect buffer and 1 μL of RNAse Inhibitor (Invitrogen). Single cell RT-qPCR was then performed using the protocol ADP41 developed by Fluidigm. Ninety-two cells (40 killers and 44 non killers) were assayed, along with bulk samples of 10, 100 and 1000 cells, along and with no-cell and no-RT controls. The panel of 95 genes included transcripts genes relevant to T cell activation, signaling and gene regulation, and was designed and manufactured by Fluidigm D3 Assay DesignD3. For data analysis, we first extracted Log 2Ex value by subtracting Ct values from a threshold of 29. We then excluded data from i) cells that had less than 40% of genes that were amplified and had a mean of Log 2Ex out of the range of population mean±3SD and from ii) genes that were amplified in <10% of cells. Post-process analysis was done using Excel (Microsoft), Prism (GraphPad), MeV49, STrenD (https://github.com/YanXuHappygela/STrenD-release-1.0) and Genemania webtool (http://www.genemania.org/).

### Example 7

Statistical analysis was done using paired or nonpaired t-tests to determine P values between groups. If more than 2 groups were compared, ANOVA was used followed with corrected post-hoc t-tests.

### Example 8

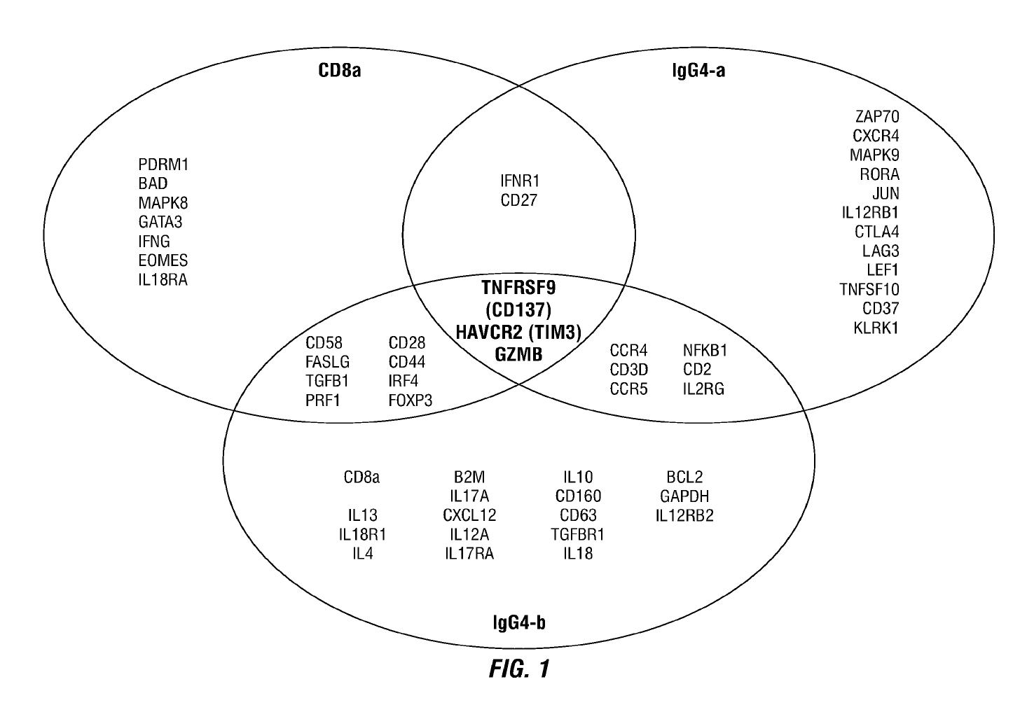

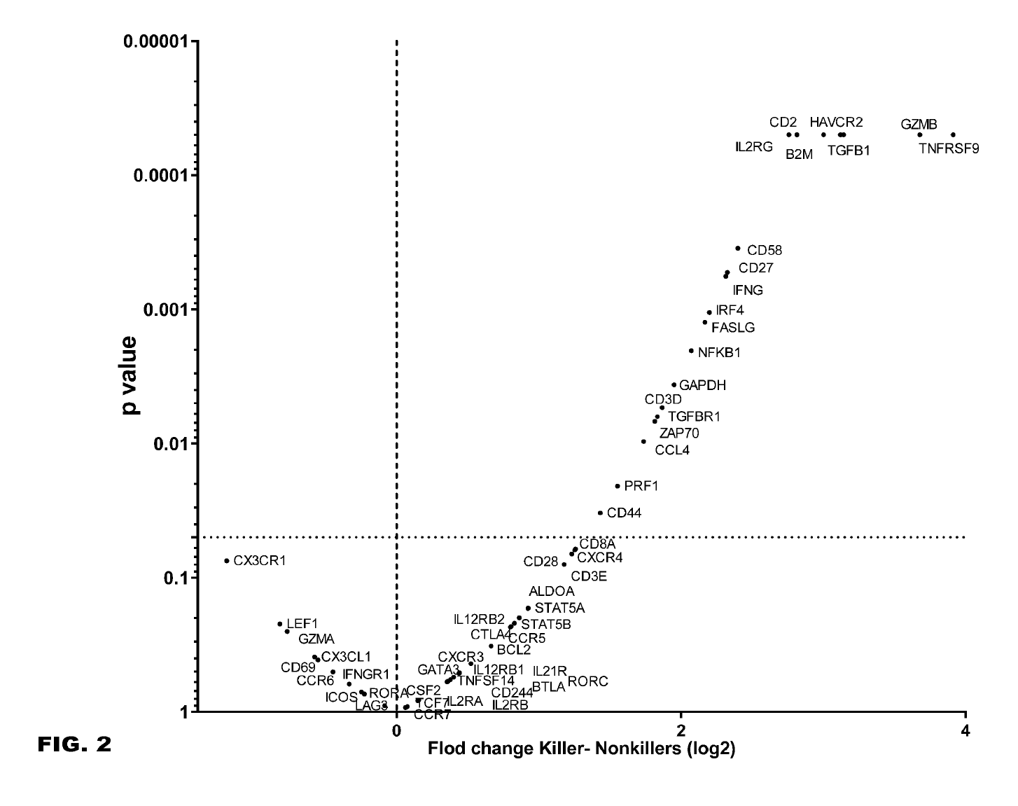

We have studied CD19-specific CAR⁺ T cells functions was assessed with CD19 expressing target cells and the gene expression profile of cytotoxic cells compared to noncytotoxic cells during a time lapse experiment of 4 h was performed. Out of 3 healthy donor CAR T cell batches, several genes were found to be upregulated in cytotoxic CAR+ T cells as compared to non-cytotoxic CAR+ T cells (FIGS. 1 and 2). Notably, CD137 mRNA transcript was found to be upregulated in all the 3 donors tested.

### Example 9

Flow cytometry based screening of candidate proteins found by transcriptome profiling was performed, and an overall homogeneity at the basal level of activation was observed (FIG. 3). In contrast, after 4 h of antigen-specific stimulation, CD137 was significantly upregulated at the surface of the CAR+ T cells (FIGS. 4 and 5).

### Example 10

CD137 was overexpressed along with CD69 activation marker and along with CD107 a degranulation marker (FIG. 5).

### Example 11

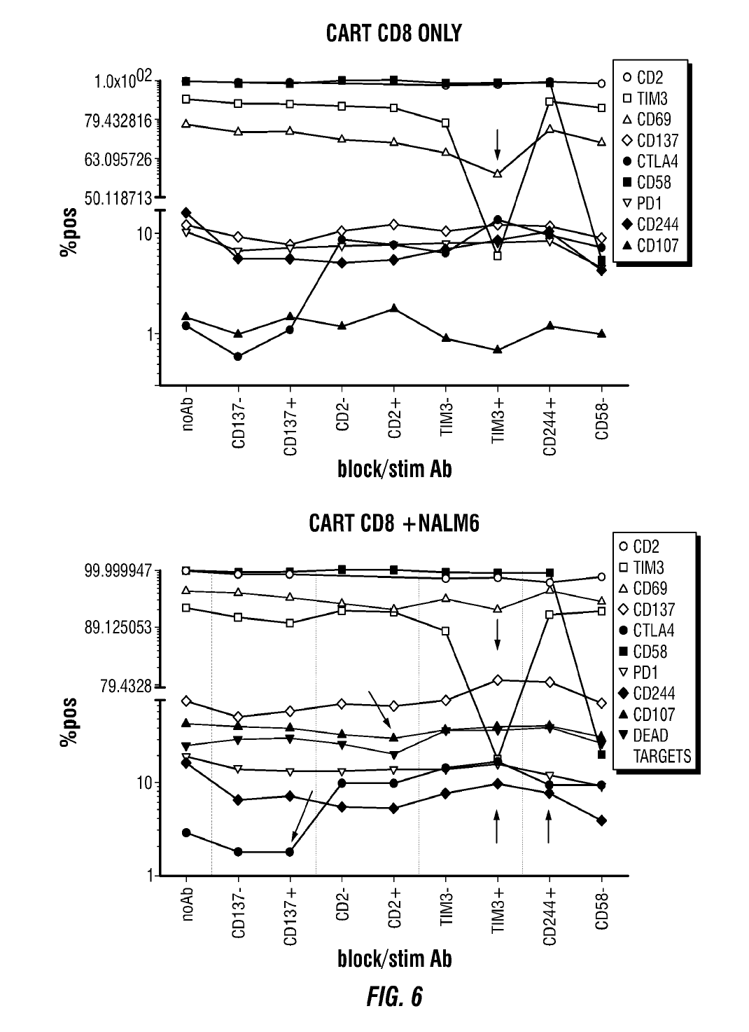

Furthermore, CD137 was stimulated by co-stimulating CAR+ T cells with CD137L before and during incubation with target cells. At the bulk level, CD137 targeting delivered by CD137L keeps TIM3 and CTLA4 at low levels as compared to no antibody control and all other receptor triggering treatments. In contrast, TIM3 targeting decreases CD69 expression and increases CD137 expression (FIG. 6).

### Example 12

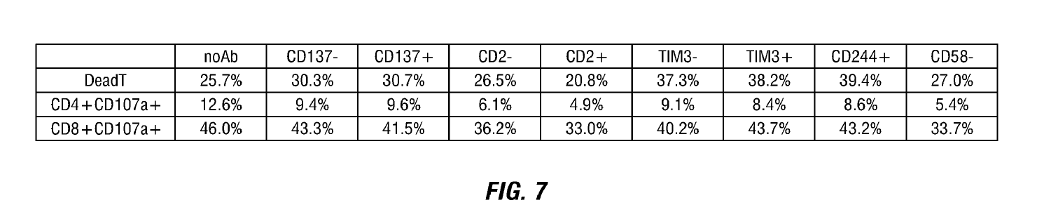

When considering cytotoxicity, CD137, TIM3, and CD244 targeting was found to lead to an increase in percentages of dead targets and at the same time, an increase in percentages of degranulating CAR+ T cells, which is consistent with higher cytotoxicity, as compared to no antibody control and all other receptor triggering treatments (FIG. 7).

### Example 13

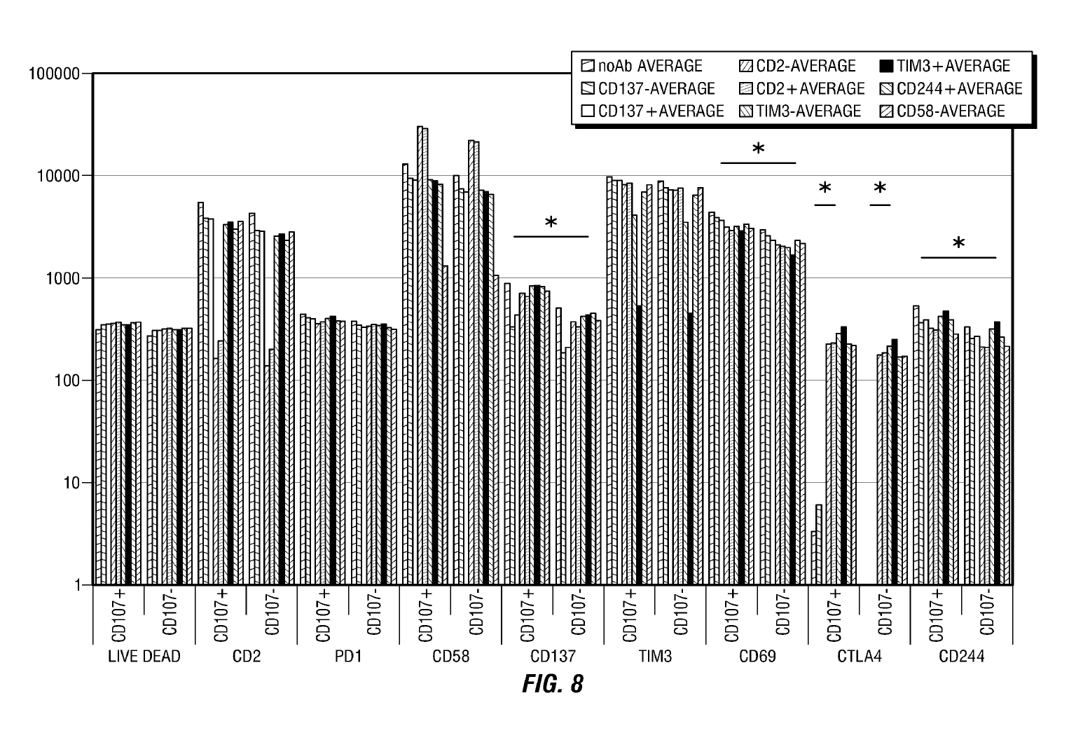

CD137 was upregulated on degranulating (CD107a+) cells (FIG. 8), suggesting that CD137 was expressed at higher levels on cytotoxic CAR⁺ cells.

The embodiments described herein are to be construed as illustrative and not as constraining the remainder of the disclosure in any way. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A method of enhancing chimeric antigen receptor expressing T cell function comprising:
activating the chimeric antigen receptor expressing T cells;
determining the differential expression of at least one molecule on the chimeric antigen receptor T cells; and
targeting the at least one molecule, wherein the at least one molecule is differentially expressed upon activation of the chimeric antigen receptor expressing T cells.

2. The method of claim 1, wherein the step of targeting the at least one differentially expressed molecule comprises targeting at least one secondary molecule upstream of the differentially expressed molecule.

3. The method of claim 1, wherein the step of targeting the at least one differentially expressed molecule comprises targeting at least one secondary molecule downstream of the differentially expressed molecule.

4. The method of claim 1, wherein the step of targeting the at least one differentially expressed molecule comprises targeting at least one secondary molecule that is a functional homolog of the differentially expressed molecule.

5. The method of claim 1, wherein the activation of the chimeric antigen receptor expressing T cells comprises engagement of the chimeric antigen receptor with its corresponding target antigen.

6. The method of claim 1, wherein the step of determining the differential expression of the at least one molecule comprises using RNA-sequencing, Nanostring analyses, RNA-FISH, microarray analysis, flow cytometry, mass cytometry, western blotting, protein staining, and microscopy.

7. The method of claim 1, wherein the step of targeting the at least one differentially expressed molecule comprises using a protein, antibody, RNA, or a small molecule.

8. The method of claim 7, wherein the small molecule is a FDA approved drug.

9. The method of claim 1, wherein the at least one differentially expressed molecule is a co-stimulatory receptor molecule expressed on the chimeric antigen receptor expressing T cells.

10. The method of claim 9, wherein the co-stimulatory molecule is selected from a group consisting of CD2, OX40, CD137, CD27, CD28, GITR, CD40 and CD30.

11. The method of claim 8, wherein the step of targeting comprises contacting an agonist of the co-stimulatory receptor molecule with the chimeric antigen receptor expressing T cells.

12. The method of claim 1, wherein the chimeric antigen receptor encodes for a nucleic acid sequence comprising:
an antigen binding domain;
a transmembrane domain;
a co-stimulatory domain; and
a CD3 zeta signaling domain.

13. The method of claim 1, wherein the antigen recognized by the antigen binding domain of the CAR comprises CD19, CD20, CD22, ROR1, mesothelin, human endogenous retroviruses, human immunodeficiency viruses, cancer-associated Tn glycoform of MUC1, EGFRVIII, GD-2, CD33/IL3Ra, PSMA, c-Met, and Glycolipid F77, or any combination thereof.

14. The method of claim 1, wherein the chimeric antigen receptor expressing T cells are effective for treating B cell malignancy, CLL, B-ALL, Leukemia, Lymphoma or solid tumors.

15. The method of claim 14, wherein the solid tumors are selected from a group consisting of breast cancer, prostate cancer, bladder cancer, soft tissue sarcoma, lymphomas, esophageal cancer, uterine cancer, bone cancer, adrenal gland cancer, lung cancer, thyroid cancer, colon cancer, glioma; liver cancer, pancreatic cancer, renal cancer, cervical cancer, testicular cancer, head and neck cancer, ovarian cancer, neuroblastoma, and melanoma

16. The method of claim 1, wherein the at least one differentially expressed molecule is an immunoinhibitory receptor molecule expressed on the chimeric antigen receptor expressing T cells.

17. The method of claim 16, wherein the immunoinhibitory receptor molecule is selected from a group consisting of CTLA4, PD1, LAGS, TIM3, BTLA or CD244, LIGHT.

18. The method of claim 16, wherein step of targeting comprises contacting an antagonist of the immunoinhibitory receptor molecule with the chimeric antigen receptor expressing T cells.

19. The method of claim 1, wherein the activation of the chimeric antigen receptor expressing T cells is measured by assessing chimeric antigen receptor T cell-mediated cytotoxicity, cytokine secretion, cell survival, phenotypic markers, calcium signaling, glycolytic activation, and cell proliferation.

20. The method of claim 19, wherein the phenotypic markers assessed comprise CD25, CD69, CD137, or CD154.

21. The method of claim 19, wherein the step of activation of the chimeric antigen receptor expressing T cells comprises coculturing with autologous tumor cells, cell lines, plate bound antibody against CD3, or beads coated with antibody against CD3/CD28.

22. The method of the claim 21, wherein the activation is measured in the presence of immunosuppressors including TGFβ, IL-10, adenosine, kynurenine, lactate, regulatory T cells, tumor derived macrophages, myeloid derived suppressor cells, tumor associated neutrophils.

23. The method of claim 22, wherein the activation is measured under nutrient starvation including glucose limitation, addition of oncometabolites, and amino acid limitation.

24. A method of treating a tumor in a subject in need thereof comprising:
obtaining chimeric antigen receptor expressing T cells expressing a chimeric antigen receptor targeting at least one tumor associated antigen;
administering to the subject an infusion of the chimeric antigen receptor expressing T cells;
determining the differential expression of at least one molecule on the chimeric antigen receptor T cells; and
targeting the at least one molecule, wherein the molecule is differentially expressed upon activation of the chimeric antigen receptor expressing T cells.

25. The method of claim 24, wherein the activation of the chimeric antigen receptor expressing T cells comprises engagement of the chimeric antigen receptor with its corresponding target antigen expressed by the tumor.

26. The method of claim 24, wherein the step of determining the differential expression of the at least one molecule comprises using RNA-sequencing, microarray analysis, flow cytometry, Nanostring analyses, RNA-FISH, mass cytometry, western blotting, protein staining, and microscopy.

27. The method of claim 24, wherein the step of targeting the at least one molecule comprises using a protein, antibody, RNA, or a small molecule.

28. The method of claim 27, wherein the small molecule is a FDA approved drug.

29. The method of claim 24, wherein the at least one molecule differentially expressed is a co-stimulatory receptor molecule expressed on the chimeric antigen receptor expressing T cells.

30. The method of claim 29, wherein the co-stimulatory molecule is selected from a group consisting of CD2, OX40, CD137, CD27, CD28, GITR, CD40 and CD30.

31. The method of claim 30, wherein the step of targeting comprises contacting an agonist of the co-stimulatory receptor molecule with the chimeric antigen receptor expressing T cells.

32. The method of claim 32, wherein the tumor associated antigen comprises CD19, CD20, CD22, ROR1, mesothelin, cancer-associated Tn glycoform of MUC1, EGFRvIII, human endogenous retroviruses, human immunodeficiency viruses, GD-2, CD33/IL3Ra, PSMA, c-Met, and Glycolipid F77, and any combination thereof.

33. The method of claim 32, wherein the chimeric antigen receptor expressing T cells are effective for treating B cell malignancy, CLL, B-ALL, Leukemia, Lymphoma or solid tumors.

34. The method of claim 33, wherein the solid tumors are selected from a group consisting of breast cancer, prostate cancer, bladder cancer, soft tissue sarcoma, lymphomas, esophageal cancer, uterine cancer, bone cancer, adrenal gland cancer, lung cancer, thyroid cancer, colon cancer, glioma; liver cancer, pancreatic cancer, renal cancer, cervical cancer, testicular cancer, head and neck cancer, ovarian cancer, neuroblastoma, and melanoma.

35. The method of claim 24, wherein the at least one molecule differentially expressed is an immunoinhibitory receptor molecule expressed on the chimeric antigen receptor expressing T cells.

36. The method of claim 35, wherein the immunoinhibitory receptor molecule is selected from a group consisting of CTLA4, PD1, LAGS, TIM3, BTLA or CD244, LIGHT.

37. The method of claim 35, wherein step of targeting comprises contacting an antagonist of the immunoinhibitory receptor molecule with the chimeric antigen receptor expressing T cells.
